(19) United States
(12) Patent Application Publication
(10) Pub. No.: US 2024/0371471 A1
(43) Pub. Date: Nov. 7, 2024

(54) SYSTEMS AND METHODS FOR DETECTING PATHOGENS IN MEDICAL SAMPLES AND DRUG RESISTANCE ANALYSIS THEREOF

(71) Applicant: Phoenix Spectroscopy Inc., Boca Raton, FL (US)

(72) Inventor: Yakov Herskowitz, Boca Raton, FL (US)

(73) Assignee: Phoenix Spectroscopy Inc., Boca Raton, FL (US)

(21) Appl. No.: 18/656,528

(22) Filed: May 6, 2024

Related U.S. Application Data
(60) Provisional application No. 63/464,472, filed on May 5, 2023.

(51) Int. Cl. G16B 35/20; G01N 21/35; G16H 10/40; G16H 20/10

(52) U.S. Cl. CPC G16B 35/20; G01N 21/35; G16H 10/40; G16H 20/10; G01N 2021/3595

(57) ABSTRACT

Systems and methods of the present disclosure are provided for detecting the presence of a pathogen or living organism within a sample. FT-IR is used to determine an absorption profile of the sample, which is used to determine the presence of the pathogen in the sample and determine a concentration of the pathogen in the sample. The systems and methods are further configured for detection and treatment of antibiotic resistance genes within the pathogen detected, using the absorption profile of the sample and the absorption profile of the pathogen.

### CLAIM OF PRIORITY

This application claims priority from U.S. Provisional Patent Application No. 63/464,472 filed May 5, 2023, the contents of which are incorporated herein by reference.

### FIELD OF INVENTION

The present invention is directed to systems and methods for medical diagnostics. More specifically, the present disclosure is directed to systems and methods for detecting infection, for example, bacteria, viruses, or fungi using Mid-infrared (MIR) spectroscopy.

### INTRODUCTION

Antimicrobial resistance is a global health and development threat. It requires urgent multisectoral action to achieve sustainable development goals.

More than 2.8 million antimicrobial resistance infections occur in the U.S. each year, and more than 35,000 people die as a result. When bacteria such as Clostridioides difficile (“C. difficile”), a bacterium causing potentially lethal symptoms such as diarrhea, becomes resistant to antibiotics, the results can be deadly. In the United States alone, there were more than 3 million infections, wherein 48,000 infections resulted in death. Moreover, according to the CDC, at least 33% of all the antibiotics prescribed in an outpatient setting, 60% of all the antibiotics prescribed in a hospital setting, and 70% of all the antibiotics prescribed in a skilled nursing facility (SNF) setting each year are incorrectly prescribed. Thus, the prescription of antibiotics has become one of the largest contributors to the rising threat of antimicrobial resistance. A lack of quick and accurate diagnostics, in conjunction with a patient's desire to receive immediate treatment, has directly contributed to said mis-prescribed antibiotics. In effect, the main cause of antibiotic resistance is improper or overuse of antibiotics, wherein said use fortifies bacteria resistant to said antibiotics. As a result, the overuse of antibiotics increases the prevalence of antibiotic resistant bacteria.

The frivolous dissemination of antibiotics increases the probability that bacteria will develop a resistance to said antibiotics. In U.S. hospitals, nearly 60% of patients receive antibiotics during their stays. Given the volume of inpatient antibiotic prescriptions, it is critical that hospitals minimize inappropriate antibiotic use. Furthermore, in addition to contributing to resistance, the overuse of antibiotics can lead to adverse events, including allergic reactions, which can range in symptoms from minor rashes to life-threatening illnesses such as C. difficile infections. Misuse of antibiotics can cause or trigger another infection, such as C. difficile, aside from the cause of antimicrobial resistance (AMR), which is also caused by treatment with the incorrect antibiotics. The over-prescription and mis-prescription of antibiotics, means that in the future there is the potential that antibiotics will lose their efficacy, ultimately leading to the proliferation of deadly bacterial infections. However, if healthcare providers decrease antibiotic proliferation, antibiotic efficacy may increase, and bacterial resistance may decrease.

Accordingly, it would be desirable to provide healthcare professionals with systems and methods capable of quickly and accurately diagnosing patients. Yet further, it would be desirable to provide systems and methods with high specificity and low costs to ensure healthcare professionals properly prescribe antibiotics. It would be desirable to diagnose and prescribe quickly and accurately, preventing the infection from spreading further. It would be further desirable to reduce septic related hospitalizations, which is the leading cause for returns to hospitals according to Centers for Medicare & Medicaid Services (CMS) and is responsible for 10% of the returns to hospitals.

### SUMMARY

This Summary is provided to introduce a selection of concepts in a simplified form that are further described below in the Detailed Description. This Summary is not intended to identify key features or essential features, nor is it intended to limit the scope of the claims included herewith.

Provided may be a method for detecting bacterial, fungal, or viral infections and associated resistance profiles in the pathogens detected, the method comprising the steps of establishing qualitative factors of a pathogen in a sample; and quantifying the colonization of the pathogen, wherein, scientifically, the method contemplates the quantity of pathogen present. However, in various embodiments, for example in cases where a patient is already on an antibiotic, the colonization may not play an as important role in determining the cause of the infection, instead it may lie with the treating physician to make such a decision. Thus, in such an instance, the method may provide the treating physician with the information. The method may further comprise the steps of determining the presence of a gene based on a pathogen structure; detecting a pathogen in the sample utilizing an artificial intelligence model; differentiating the pathogen structure from a non-pathogen structure in the sample utilizing the artificial intelligence; reporting data produced from differentiating the pathogen structure from the non-pathogen structure; and distributing the data to healthcare professionals.

Provided may be method for detecting pathogens in a sample comprising the steps of processing a sample using MIR spectrometry to identify sample spectra data and detecting a presence of a pathogen in the sample spectra data, wherein the presence of the pathogen is detected according to a method comprising the steps of: identifying the pathogen in the sample, wherein the pathogen is identified when a concentration of the pathogen in the sample is over a level of detection (LOD); identifying a volume of the pathogen in the sample, wherein the volume is a level of quantification (LOQ) of the pathogen; and determining the presence of a resistant gene in the pathogen. The method may further include the step of generating a report comprising the pathogen detected and the presence of resistant genes in the sample.

In an embodiment, the MIR spectrometry is FT-IR spectrometry.

In an embodiment, identifying the pathogen in the sample comprises comparing the sample spectra data with a plurality of pathogen profiles, each of the plurality of pathogen profiles corresponding to a known pathogen and comprising spectra data corresponding to the known pathogen. In an embodiment, the step of comparing the sample spectra data with the plurality of pathogen profiles comprises determining a best fit pathogen profile to the sample spectra data. In an embodiment, identifying the volume of the pathogen in the sample comprises determining a concentration of the pathogen in sample.

In an embodiment, when the presence of the resistant gene is determined, the method further comprises the step of correlating a protein structure of the resistant gene with a database to determine an antibiotic resistance of the pathogen.

Provided may be a method for detecting pathogens in a sample comprising the steps of processing a sample using an FT-IR spectrometer to identify sample spectra data, wherein the sample spectra data comprises an absorption spectrum of the sample and detecting a presence of a pathogen in the sample spectra data, wherein the presence of the pathogen is detected according to a method comprising the steps of identifying the pathogen in the sample, wherein identifying the pathogen in the sample comprises comparing the sample spectra data with a plurality of pathogen profiles, stored in a database, each of the plurality of pathogen profiles corresponding to a known pathogen and comprising spectra data corresponding to the known pathogen; identifying a concentration of the pathogen in the sample; and determining the presence of a resistant gene in the pathogen from the absorption spectrum of the sample and the database to determine an antibiotic resistance of the pathogen. The method may further comprise the step of generating a report comprising the pathogen detected and the presence of resistant genes in the sample.

In an embodiment, the absorption spectrum of the sample is a compared to an absorption spectrum of the known pathogen in the plurality of pathogen profiles to determine a best fit, by comparing any of a frequency, amplitude, or spread of the absorption spectrums. In an embodiment, the concentration of the pathogen in the sample corresponds to any of a high colonization, a medium colonization, or a low colonization of the pathogen.

In an embodiment, when the concentration of the pathogen is the high colonization, the report comprises to a recommendation of treatment. In an embodiment, the high colonization corresponds to at least 100,000 CFU per ml, the medium colonization corresponds to 50,000-100,000 CFU per ml, and the low colonization corresponds to 10,000-50,000 CFU per ml.

In an embodiment, when the presence of the resistant gene is determined, the method further comprises correlating a protein structure of the resistant gene with a database to determine an antibiotic resistance of the pathogen.

In an embodiment, wherein identifying the pathogen comprises identifying the presence of the pathogen greater than a level of detection (LOD).

In an embodiment, the step of identifying the concentration of the pathogen in the sample further comprises comparing the sample spectra data with the plurality of pathogen profiles, stored in the database, wherein each of the plurality of pathogen profiles comprise concentration-specific spectra, wherein each of the concentration-specific spectra are determined by using the FT-IR spectrometer with reference samples of varying concentrations.

In an embodiment, the plurality of pathogen profiles comprises pathogen profiles of subtypes.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates a block diagram of a distributed computer system that can implement one or more aspects of the present invention.

FIG. 2 illustrates a block diagram of an electronic device that can implement one or more aspects of the present invention.

FIG. 3A illustrates an embodiment of a sample analysis pathway.

FIG. 3B illustrates an embodiment of a sample analysis pathway.

FIG. 4A is a graph illustrating an example of data collected from an embodiment of the analysis pathway.

FIG. 4B illustrates another graph of the example illustrated in FIG. 4A.

FIG. 5 illustrates an embodiment of a qualitative and the quantitative data output pathway.

FIG. 6 illustrates an embodiment of a system and method analyzation flowchart.

FIG. 7 illustrates an embodiment of a user interface and example report.

FIG. 8A illustrates an example of spectra data for multiple pathogens.

FIG. 8B is a dendrogram for the spectra data shown in FIG. 8A.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

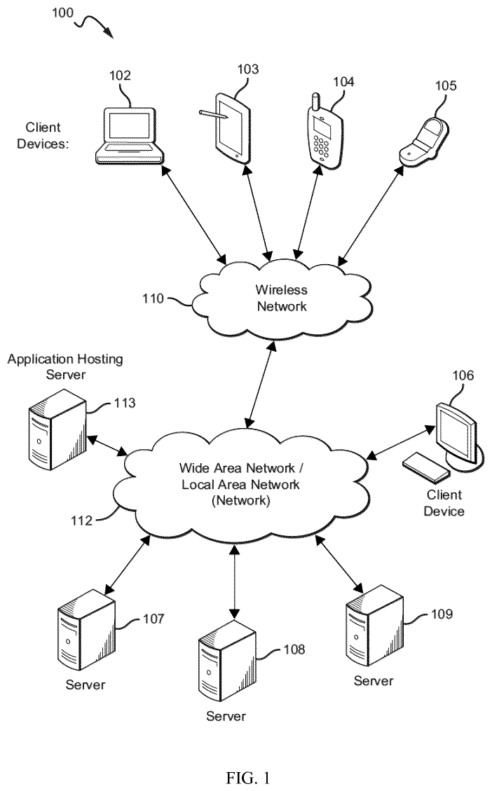

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

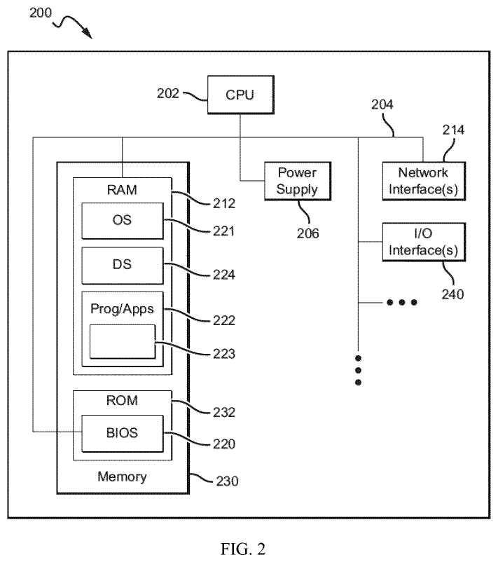

FIG. 2 illustrates a block diagram of an electronic device 200 that can implement one or more aspects of an apparatus, system and method for validating and correcting user information (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can case processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as IOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games, receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPV6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The invention of the present disclosure may be directed to utilization of Mid-infrared (MIR) spectroscopy technology as applied to pathogen detection. Conventionally, the methods available for detection of an infection includes Culture and Sensitivity (C&S), which contains countless limitations with its inability to accurately diagnose and therefore accurately treat; or Polymerase Chain Reaction (PCR) technology, which although more accurate and capable of solving the majority of C&S limitations, includes many additional limitations. As a nonlimiting example, PCR technology may cause false detection of particular DNA/RNA fragments, improperly posing as an infection. Next-generation sequencing (NGS) has many limitations aside from its exuberant cost and lengthy turnaround time. As a non-limiting example, implementation of NGS for infection detection may result in a “shotgun” approach, which may be fatal. Moreover, the aforementioned traditional testing methods may be considered high complexity testing, which requires a laboratory in which all the samples will be processed in. In an embodiment, the systems and methods of the present disclosure may utilize MIR, wherein such technology may be available at every doctor's office, hospital, urgent care, or skilled nursing facility as a low complexity test, therefore, allowing such individuals and facilities to run MIR tests in their office, providing results within seconds at a higher accuracy than the aforementioned traditional methods.

Accordingly, as described herein, by utilizing MIR technology, the presence of a pathogen may be detected with the upmost accuracy and specificity. In various embodiments, MIR technology may provide results of qualitative and quantitative significance, as well as antibiotic resistance (sensitivities), within minutes, while accruing no or negligible cost of any consumables.

As described herein, MIR spectroscopy may traditionally be utilized for the purpose of determining food quality and/or composition. The systems and methods of the present disclosure are configured for detection of the presence of a pathogen or living organism within a sample. Yet further, the systems and methods of the present disclosure may be configured for detection and/or treatment of resistance within the pathogen detected. In an embodiment, the system may include an artificial intelligence (AI) component adapted to sift through the data and create predictive algorithms.

As described above, in relation to C&S, different growth methods are required for different pathogen types, requiring multiple tests, over various turnaround times (TAT) to develop a complete picture of the near-countless possibilities. Moreover, C&S may require viable microbes; thus, if the collection was imperfect (i.e., not a temperature-controlled environment), microbes will die and will result in no or inaccurate growth, deeming the results useless (i.e., there may be no way to know that that is what occurred). Another deficiency in C&S technology stems from the likelihood for pathogens to die in the transportation process. Significantly, C&S may not detect biofilm infections which are present in over 80% of infections. C&S may also require a great deal of time, for example, a minimum TAT of 72 hours (including sensitivity) and even several weeks for fungi. C&S technology may create extreme difficulty in detecting multiple pathogenic infections, which is true for the majority of infection cases. Also, in utilizing C&S, a treatment recommendation will be based only on pathogens detected, this can be the wrong treatment in many cases as often, other pathogens are not detected and can be resistant to the medication prescribed.

PCR technology may only detect targeted pathogens and detected antibiotic resistant genes are detected in the sample not the pathogen. Further, PCR technology may unnecessarily detect DNA/RNA fragments, resulting in false positives. Moreover, PCR technology may prove costly and time-consuming to operate.

NGS technology may not provide a true quantitative value, further, NGS technology may uncover superfluous information, making it difficult to properly determine cause of infection. NGS technology may prove costlier and more time-consuming than PCR technology.

MIR spectroscopy may solve the aforementioned problems represented in C&S, PCR, NGS, and other similar technologies. Accordingly, MIR spectroscopy and the related systems and methods disclosed herein, may provide accurate qualitative and quantitative results; may determine antibiotic resistance with high accuracy; may require no or negligible cost for consumables; and may provide results in minutes. Specifically, the MIR spectroscopy systems and methods described herein may increase efficiency (e.g., cutting costs; improving quality of care; and reshaping antibiotic administration). Further, the systems and methods described herein may increase usability (e.g., decreasing the need to send a sample to the lab).

In an embodiment, a MIR spectrometer component may be utilized in the detection and/or analysis steps. The systems and methods of the present disclosure may utilize any suitable MIR devices. For example, such suitable devices may include compact Fourier-transform infrared spectroscopy (FT-IR) spectrometers configured for biological and chemical analysis, as discussed in more details herein.

In an embodiment, the MIR spectrometer and/or component(s) thereof may be configured to receive a collected sample, wherein the extracted data may determine the presence of a pathogen in the sample. For the purposes of this disclosure, the FT-IR detections components and steps recited herein may be tailored for MIR. Yet further, recitation of MIR throughout this disclosure may interpreted as use of FT-IR.

Thus, after analysis by the MIR spectrometer, the system may provide results, wherein said results may provide a doctor or diagnostician the ability to diagnose and prescribe an accurate antibiotic within minutes, instead of days or weeks after initial consultation.

In an embodiment, the provided disclosure may describe a platform that may be capable of analyzing and interpreting the data extracted from a collected specimen (i.e., a urine sample) to determine the presence of a pathogen within the sample. The collected specimen may be any of a urine sample, protein matrix-transport media, or other suitable specimen.

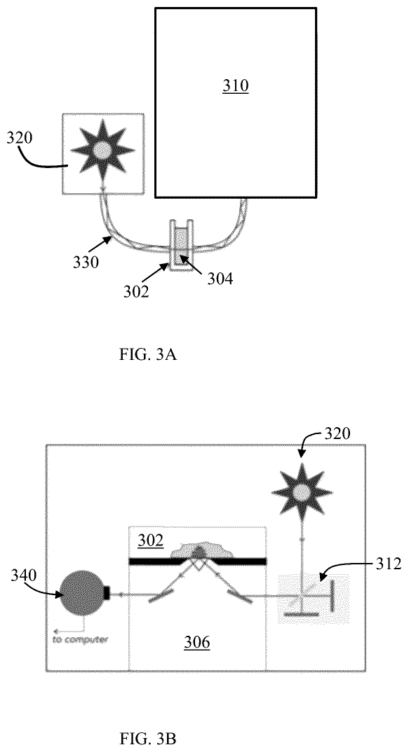

Referring to FIG. 3A, a sample 304 may be placed in the spectrometer 310. The system may include a source 320 configured to produce an electromagnetic signal. The system may further comprise a transparent fiber 330, wherein said fiber 330 is capable of transmitting the electromagnetic signal. In an embodiment, the fiber 330 is an optical fiber. Additionally, a vessel 302 containing the sample 304 may be disposed along the fiber's path. In an embodiment, the vessel 302 may be a cuvette. In another embodiment, the vessel 302 may be a microcentrifuge tube. However, the vessel 302 containing the sample 304 may include any suitable vessel alternative. In a different embodiment, the electromagnetic signal may be transmitted through an optical fiber, wherein the electromagnetic signal passes through the vessel 302 and the sample 304, creating a transformed signal.

Turning to FIG. 3B, the system may include a spectrometer 310 capable of conveying data to a computer. The spectrometer 310, may receive the transformed signal, wherein said signal may be further processed by a Michelson interferometer 312. In an embodiment, the transformed signal is received by the spectrometer 310, wherein the Michelson interferometer 312 produces an encoded signal. The encoded signal may pass through a sample chamber 306 before reaching a detector 340. The detector 340 may decode the encoded signal and transmit it to the computer for analysis. In an embodiment, the encoded signal passes through the sample chamber 306, wherein the chamber 306 transmits the encoded signal to the detector 340, the detector 340 decodes the encoded signal, creating a decoded signal, and passes the decoded signal to the computer.

In one embodiment, the spectrometer may be a Fourier transform infrared (FTIR or FT-IR) spectrometer. The FT-IR spectrometer may measure the intensity of light in the MIR region as to collect and analyze spectra in the sample. It is contemplated that, in some embodiments, the use of FT-IR spectrometry permits analysis of a sample, as opposed to a whole organism, which is otherwise required by prior art systems.

In some embodiments, the FT-IR spectrometer comprises an internal reflection element which requires a high refraction index. The internal reflection element may be an attenuated total reflection (ATR) crystal or other suitable reflection elements. An IR light may be directed through the internal reflection element and may be reflected on the internal reflection element surface. The IR light may be absorbed by the sample and, thus, may be missing in the reflected beam. As a result, the reflected beam may have a varied intensity depending on the wavelength absorbed by the sample. The reflected beam may be measured using attenuated total reflection, also known as internal reflection spectroscopy (IRS) to determine an absorption spectrum of the sample. The absorption spectrum may be interchangeably referred to as sample spectra data.

In some embodiments, the FT-IR spectrometer may further comprise an anvil, a means for moving the anvil, a pressure arm, and a pressure control interface. As a person of ordinary skill in the art will recognize, anvils are utilized with FT-IR spectrometry to study the effects of pressure on the molecular structure and properties of the sample. The anvil may comprise a material transparent to infrared to permit the IR light to pass through the sample without significant absorption or scattering. In some embodiments, pressure may be applied to the sample by the anvil to alter the molecular structure of the sample. Any of the means for moving the anvil, the pressure arm, and the pressure control interface may be utilized to position the anvil and apply pressure to the sample. Of course, other configurations of anvils are contemplated and the aforementioned is provided as a nonlimiting example only.

The testing on the sample may comprise collecting a specimen and preparing the sample for testing according to any manners that are known in the art. In some embodiments, the sample may be a liquid sample, such as urine, collected from a clean catch or from a fresh catheter. However, in other embodiments, the sample may be a solid sample, such as a wound scraping collected using sterile instruments after the wound has been debrided. The aforementioned examples and collection of samples are not considered limiting, and the sample may be a fungal scraping or any other sample to be examined.

The FT-IR spectrometer may be prepared for testing by the cleaning the internal reflection element, permitting the internal reflection element to dry, and conducting a background check. The background check may, in some embodiments, comprise calibrating the system. The background check may comprise running a calibration program on the FT-IR spectrometer. In some embodiments, the background check may determine a spectra calibration of the system, corresponding to the absorption spectrum generated by the spectrometer in the absence of a sample. Following performing the background check, the sample may be tested by the FT-IR spectrometer. In one embodiment, testing the sample may comprise placing the sample on the internal reflection element and selecting a testing program on the FT-IR spectrometer. In some embodiments, the FT-IR spectrometer may be communicatively coupled to an electronic computing device.

During testing, the sample may be scanned any number of times necessary to collect spectra data for the sample. In one embodiment, the sample may be scanned at least 50 times to collect the spectra data. For example, the sample may be scanned at least 100 times, at least 150 times, at least 200 times, or at least 250 times. In an embodiment, the sample may be scanned 256 times to determine the spectra data. The aforementioned examples are provided as nonlimiting examples, and any number of scans suitable to determine spectra data is contemplated.

Further, other methods of determining the spectra data are contemplated and the aforementioned are provided as nonlimiting examples only.

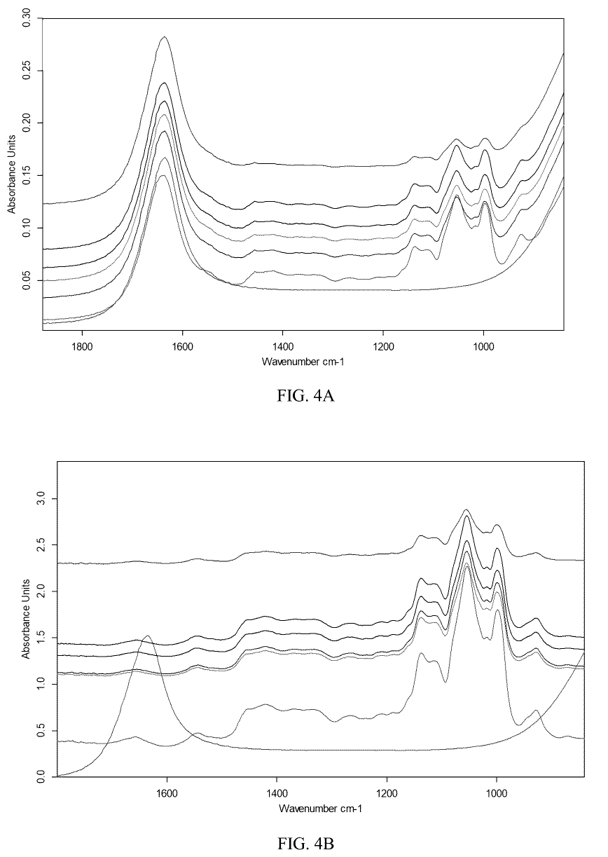

One example of the spectra data is shown in FIGS. 4A-4B, which illustrate the FT-IR spectrometer data for various samples as discussed herein.

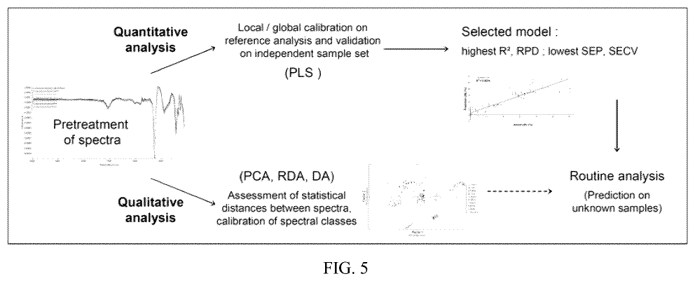

Referring to FIG. 5, the system may comprise qualitative and the quantitative data output from the spectrometer. The system may execute a quantitative analysis. The quantitative analysis may rely on a calibration of a reference analysis and/or validation of an independent sample set. In an embodiment, the calibration is a local and/or global calibration. Furthermore, the quantitative analysis may include a selected model, wherein the selected model may comprise a coefficient of determination, a relative percent difference, a lowest standard error for difference proportion, and/or a standard error of cross validation. Said quantitative analysis, may be capable of performing a routine analysis as a means for predicting characteristics of unknown samples.

The system may further execute a qualitative analysis. The qualitative analysis may include a statistical analysis, wherein the statistical analysis may assess the statistical distance between the sample spectra data and a reference. In an embodiment, the statistical analysis is a principal component analysis. In another embodiment, the statistical analysis is a redundancy analysis. In yet a further embodiment, the statistical analysis is a discriminant analysis. The qualitative analysis may be capable of performing a routine analysis as a means for predicting characteristics of unknown samples.

In an embodiment, the spectrometer may provide the composition of the specimen to determine its health properties, protein levels, product potential yield composition, antioxidant activity, and other relevant factors. Accordingly, the systems and methods described herein may determine presence of a particular organism(s), potential yield, and associated characteristics pertaining specifically to susceptibility or resistance to specific antibiotic classes, based on particular gene presence within the pathogen itself, as it may be protein based (e.g., a Protein Based Assay). In one embodiment, such an analysis differs from PCR analysis, wherein, in PCR, the technology may detect the presence of a gene in the sample, not necessarily whether it is in the pathogen.

The method of the present disclosure comprises a number of steps configured to extract meaningful information from a patient sample and derive qualitative and quantitative results of the resistant patterns in the detected pathogens.

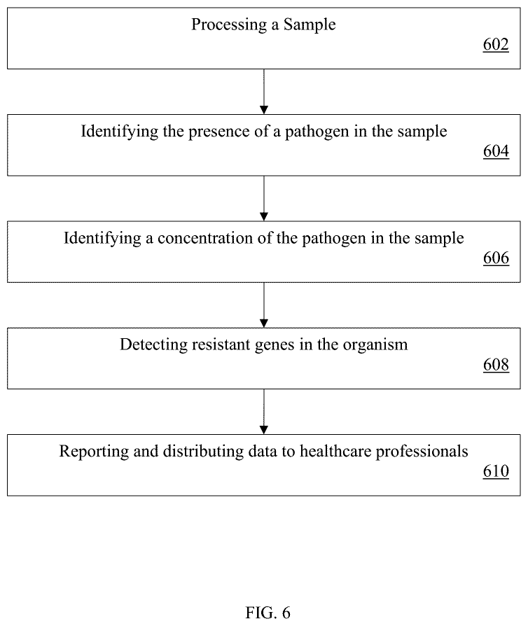

FIG. 6 illustrates an embodiment of an exemplary workflow of the method. The method comprises step 602 of processing a sample to determine sample spectra data, step 604 of identifying the presence of a pathogen in the sample using qualitative factors, step 606 of identifying a concentration of the pathogen in the sample using quantitative factors, step 608 of detecting resistant genes in the organism, and step 610 reporting and distributing data to healthcare professionals.

The step 602 of processing the sample may be any process, including, for example, the FT-IR spectrometry preparation described above.

At step 604, the qualitative factors of each individual pathogen may be established. Further, the Limit of Detection (LOD) may be determined. In determining the LOD, the system may use live organisms, which may provide a basis, as well as the defined parameters of each potentially detected individual organism. Once parameters (e.g., LOD) have been established, the method continues to step 606.

For the purposes of this disclosure, LOD determination may be performed while setting up the test. For example, the system may determine the limits of detection. For example, the system may determine a minimum concentration in which the pathogen will be detected. The LOD determination may provide threshold information, for example, if the system detected x amount per ml, based on the determined LOD data, the system may evaluate that such an amount translates to s specific number of colonies growing. Thus, the system may be configured to determine the number of colonies of said pathogen present based on LOD thresholds.

In some embodiments, the system may comprise a database comprising a plurality of pathogen profiles. Each of the pathogen profiles may comprise a spectrometry reading for at least one concentration level of the pathogen in the sample. The pathogen profiles may be locally generated or may be globally generated.

In some embodiments, the system may determine the presence of the pathogen in the sample by comparing the sample spectra data with the plurality of pathogen profiles in the database. Each of the plurality of pathogen profiles may comprise a profile signature and a pathogen that the profile signature represents. The profile signature may comprise the spectra data corresponding to the pathogen and may comprise any of an amplitude, frequency, or spread of the absorption spectrum.

In one embodiment, the plurality of pathogen profiles compared to the sample may be limited, for example by testing parameters received, to less than all of the plurality of pathogen profiles in the database. For example, the plurality of pathogens in the database may be limited to influenza strains. Of course, the plurality of pathogens may be limited according in any manner and the aforementioned are provided as nonlimiting examples only. Further, in other embodiments, the system may compare the sample spectra data to each of the plurality of pathogen profiles.

Comparing the sample spectra data may comprise a comparison of any of the frequency, amplitude, or spread of the sample with the frequency, amplitude, or spread from any plurality of pathogen profiles to identify a statistical similarity between the sample and the pathogen profile. The pathogen profile of the plurality of pathogen profiles having the best fit, and thus a high statistical similarity, is contemplated to correspond with the pathogen in the sample. Any statistical comparison method is contemplated.

At step 606, since the profile signature of the pathogen has been established, the system may determine the statistical distance between any of the spectra calibration, the sample spectra data, and the plurality of pathogen profiles. In some embodiments, the statistical distance may correspond to a specific volume of the pathogen in the sample which may correspond with the Limit of Quantitation (LOQ). For the purposes of this disclosure, a pathogen being relevant to a healthcare professional may be based on the colonization of the pathogen.

As a nonlimiting example, when a sample is to be tested in a lab, to determine if it is in fact pathogenic, the colonization may be quantified in a petri dish. Thus, at step 606, the system may promote growth of the pathogen on a dish, then, using the spectrometer the system may determine the statistical distance between any of the spectra calibration, the sample spectra data, and the plurality of pathogen profiles, which, in turn may equate to a specific volume (e.g., CFU per ml).

It is contemplated that a serial dilution may be set up in the transport media, wherein there may be a specific amount of the pathogen in the media. The system may be informed of the exact concentration upon placing a specific amount into the media. In some embodiments, the use of serial dilutions may be utilized with the plurality of pathogen profiles to detect the specific amount of the pathogen in the media for comparison with the sample spectra data. It is contemplated that the use of serial dilution may be utilized to determine the LOQ between the growth of the pathogen and normal flora in the sample and may, in some embodiments, be utilized for diagnostic purposes.

FIGS. 4A and 4B illustrate an example of the spectra data for the SARS-COVID pathogen at various dilutions. FIG. 4A illustrates six samples of various concentrations of SARS-COVID pathogen and a control sample that illustrates spectra data for water only shown at the bottom of the graph. The six samples of SARS-COVID pathogens range in concentration from zero to 10%, with the highest concentration being at the top of the graph and the lowest concentration being displayed above the spectra data for water. FIG. 4B illustrates the same spectra data as FIG. 4B with the water being removed from the SARS-COVID samples. In some embodiments, the removal of water spectra data from the pathogen samples may improve the ability to detect pathogens and determine the pathogen concentration in the sample.

Thus, when the spectra is measured, the system may determine what constitutes a high colonization (for example, 100,000 CFU per ml), a medium colonization (for example, 50,000-100,000 CFU per ml), and a low colonization (for example, 10,000-50,000 CFU per ml) of the pathogen. The colonization level may provide a quantitative representation of the sample that may be used to diagnose and/or determine a treatment. For example, a low colonization may correspond to instances where the patient is asymptomatic and does not require treatment whereas high colonization represents an active infection and the presence of symptoms that requires treatment. Accordingly, determinations of a low colonization may be utilized to treat the patient proactively, before more serious symptoms emerge. Similarly, determination of a high colonization may be utilized to treat the patient more aggressively or in a manner conducive for mature illness.

In some embodiments, the LOQ may be utilized to filter out noise in the system caused by the normal flora to identify the presence of the pathogen in the sample. It is contemplated that this may reduce errors in the system and may permit a more accurate analysis of the concentration of pathogens.

It is contemplated that the use of FT-IR may permit the differentiation between similar pathogens in the sample. In one example, the use of FT-IR spectrometry may be utilized to detect differences between similar pathogens, for example differences between the Influenza A and Influenza B viruses.

In an embodiment, the system may utilize the spectrometer data to detect genetic or structural differences. The genetic or structural differences may correspond to differences between the sample spectra data and the plurality of pathogen profiles. For example, a difference between a frequency, curve, and/or amplitude of the spectrometry data may correspond to a nucleotide difference. As a nonlimiting example, both Influenza A and B viruses are RNA viruses belonging to the Orthomyxoviridae family, however, they have different genetic compositions. For the purpose of this example, Influenza A viruses have a segmented genome consisting of eight RNA segments, while Influenza B viruses have a segmented genome with only seven RNA segments. Thus, the spectral profiles may differ due to the genetic and structural differences between the types of Influenza virus (i.e., A versus B). Yet further, the FT-IR methodology described herein may be utilized to determine the presence and/or quantity of subtypes of a given virus or virus variant. As a nonlimiting example, Influenza A viruses may be classified into subtypes based on the combination of two surface glycoproteins, hemagglutinin (HA) and neuraminidase (NA). These subtypes may combine in various ways to manifest different subtypes (e.g., H1N1). Thus, further, the FT-IR methodology described herein may be utilized to detect the presence and/or the quantity of a given subtype of a given variant of a given virus. Accordingly, by generating reference samples of the various subtypes, variants, and viruses, FT-IR profile results may be compared to these reference samples to determine the presence of the subtype, variant, or virus, and, by comparing to the dilution levels within the reference samples, even the quantity and/or colonization level.

In some embodiments, the system may be operative to identify the presence of specific genes in the pathogen. The presence of specific genes in a pathogen may render the pathogen with the ability to resist a specific treatment (e.g., a particular antibiotic).

As a nonlimiting example, the mecA gene in a staph infection will render the infection resistant to Methicillin. Accordingly, the system may comprise a curated list or database of genes which correlate to specific antibiotic, or other, resistances. In various embodiments, the systems and methods described herein may utilize a prebuilt or external database, wherein such a database and information thereof may be utilized for detecting a particular mutation in said pathogen based on the protein structure of the pathogen detected (for example, unlike PCR which is detecting the presence of a specific gene in the sample, not necessarily the actual pathogen). Of course, other genes may be detected in the pathogen and the aforementioned are provided as a nonlimiting example only.

Thus, in step 608, the system may detect these genes based on the protein makeup and structure of the pathogen. In such an embodiment, the system and, specifically, components described in FIGS. 1-2 and 3A-3B may compare extracted information (i.e., protein and structure) and perform similarity analysis or other comparisons, via a gene-resistance-correlation database, to determine the incident genes and/or resistance profiles. As a non-limiting example, each gene may have a protein structure FT-IR (a protein-based assay), for example, generating said information based on the protein bonds. Accordingly, once a pathogen is detected, based on the sample spectra data, the system may determine the protein composition of the sample. Further, in some embodiments, each type of unique profile signature for each individual pathogen and/or resistant gene may be built based on LOD and LOQ determination methods.

Prior method steps may identify parameters (i.e., Qualitative-LOD) and measure volume (i.e., Quantitative-LOQ). In some embodiment, an AI component may be trained. For example, in training, the AI component may receive 3D model representations of the sample spectra data, wherein the AI component may analyze the 3D model representations from all angles, such that the trained AI component may separate the different profile signatures that have been created by sample and select the one or more unique signatures which the AI component has been taught to select. In an embodiment, the AI component may be trained on how to detect the pathogens and their volumes based on the profile signature obtained from the clean samples. These clean samples may, for example, be non-pathogenic samples of the same medium. As a non-limiting example, after the AI component has been trained, the AI component may be tasked to uncover the data when the pathogen is spiked into a clean urine sample. Accordingly, the AI component may learn to account for the background noise and superfluous data created by the urine, human cells, etc. Thus, the AI component may detect and quantify the wanted information. The wanted information may, in some embodiments, be the presence of the pathogen in the sample at or above a specific level, for example the LOQ.

It is contemplated that the use of FT-IR spectrometry may permit the accurate detection and differentiation of pathogens, whereas traditional NIR spectrometry can only predict whether a pathogen might be present in the sample. Thus, the FT-IR spectrometry is capable of accurately detecting an actual occurrence of a pathogen in the sample and permits an accurate diagnosis of the patient without requiring the use of AI or machine-learning technology required in prior art systems to predict the likelihood that a pathogen is present. However, in further embodiments, AI or machine-learning technology may be utilized in conjunction with the FT-IR methodology described herein to induce improved results not feasible with NIR or other traditional technologies.

While reference is made to the pathogen throughout, the system and method may be utilized to detect multiple pathogens within the sample. When detecting multiple pathogens in a sample there are a great number of possible combinations while targeting a relatively small number of pathogens. For example, in an embodiment wherein sixteen pathogens are targeted there are 10e¹⁵ pathogen combinations that could be detected. The systems and methods described herein may be configured to sort this disarray of data.

In an embodiment, a trained AI component may examine the data, in an attempt to locate the specific data points of each pathogen in a live sample (i.e., human sample) and correlate it with the specific mutations that will ultimately produce the desired results. Accordingly, the AI component may be adapted to this process. In an embodiment, said AI component may be able to learn how to differentiate between background noise caused by the human cells and other possible interferences from actual relevant data. In an embodiment, the AI component, having examined the data, will differentiate the human cells in a live sample from the pathogen.

However, in another embodiment, the determination of multiple pathogens in the sample may occur in the absence of AI or other machine-learning. For example, such a determination may be made via automated statistical modeling or manual interpretation.

In an embodiment, after identifying qualitative and quantitative results in addition to the resistant patterns in the pathogens), the relevant results may be distributed in a report 700.

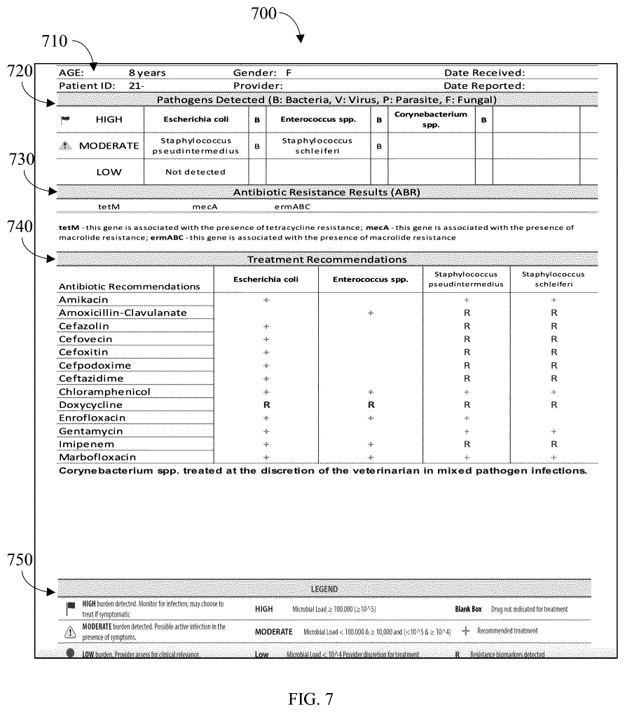

One example of the report 700 is illustrated in FIG. 7 and may be displayed on a user interface. The report 700 may be generated and/or displayed with the hardware shown in FIGS. 1-2 and described above. Said interface may display patient data 710. Moreover, the report 700 may comprise detected pathogens 720, antibiotic resistance of said pathogens 730, and/or treatment recommendations 740 that are displayed. In an embodiment, the patient data 710 may comprise any of a patient's age, ID, Gender, Primary Care Provider, sample receipt date, and/or sample report date. In another embodiment, the user interface, after the systems and methods have been ran, will display the pathogens detected 720 in the patient's sample. In a further embodiment, the user interface, after the pathogens 720 have been detected, may display a list of antibiotics that said pathogens 720 are resistant to as the antibiotic resistance of said pathogens 720 on the report 700. In yet a further embodiment, the antibiotics said pathogens are resistant to may inform treatment recommendations, which may be displayed to guide healthcare professionals in curating a treatment plan.

In an embodiment, in supplement or as a replacement of the “Antibiotic Resistance Results” section 730, the report may comprise data based on FT-IR data. For example, in one embodiment the report may comprise FT-IR data relevant to the structure of the pathogen and not the expression of a particular gene.

In the embodiment illustrated, the report 700 may comprise a legend 750 that may be utilized to interpret any of the information within the report.

It is contemplated that presenting the report 700 in this, or a similar, manner may manipulate historically complex data such that it is easy to read. This may reduce the time required to interpret test results, permitting quicker treatment, and reducing the potential for interpretation errors.

In an embodiment, the MIR device may include or may be in communication with one or more computerized devices that comprise computer-executable instructions. The computer-executable instructions, when executed by the MIR device or associated computerized devices, may cause the MIR device or associated computerized devices to perform one or more of the steps described above and herein. The MIR device may provide a spectrum of the matrix and protein structure. The computer-executable steps may, first, identify the component through classification, for example, via principle component analysis or a comparison of the spectral signature. The computer-executable instructions may comprise the ability to quantify said materials. The FT-IR device may implement this through beers law treatment or through Partial Least Squares (PLS) regression/Chemometrics. In such instances, with the matrices discussed herein, the chemometrics module may be utilized for said complex samples.

The computer-implemented instructions may be configured to implement each of the method steps described herein. The computer-implemented instructions may be configured to determine the spectral signature and the limit of detection/quantitation. The computer-implemented instructions may be configured to determine the aforementioned factors in the matrices that are of interest (for example, SARS-Cov2). In an embodiment, the sampling may be one or more drops onto the diamond crystal of the ATR.

The aforementioned disclosure may address the issue of turnaround time for clinical diagnostics, by enabling healthcare professionals to completely eliminate the pitfalls associated with empirical treatment.

A second issue the aforementioned disclosure may address is removing guesswork from medicine. Currently, the state of art relies on technologies that are limited by guesswork and trial and error procedures. Applying FT-IR spectroscopy to diagnostics may allow healthcare professionals, in a variety of settings, to accurately detect viral, fungi, as well as bacterial infections, and determine the bacterium's resistance to antibiotics. As a result, the aforementioned systems and methods may enable healthcare professionals to accurately prescribe medications and plot a patient's course of treatment.

A third issue the aforementioned disclosure may address is the costs associated with current clinical diagnostic practices. Traditional and contemporary clinical diagnostic techniques are currently cost-prohibitive for healthcare professionals outside of a hospital. Even in a hospital setting, healthcare professionals are limited to running full PCR and other high-volume tests. Exploiting FT-IR Spectroscopy may be an economic alternative for healthcare professionals by eliminating costs associated with consumable products. According to the OIG report on RTH (Return To Hospital), 27% of patients return to the hospital within 30 days because of an infection. Thus, the system and methods described herein (for example, as implemented in a tabletop unit) may completely eliminate the 27% RTH caused by infections. Further, such an implementation may translate to savings of antibiotic resources, antibiotic costs, treatment resources, and treatment costs that would otherwise be needed due to wrongful prescriptions.

The method described herein may comprise comparing sample spectra (e.g., those determined via the MIR analysis steps described above) to a reference spectra (e.g., those of known pathogens and/or pathogen concentrations) utilizing various statistical methodologies. In one embodiment, the method includes the step of preprocessing the sample spectra and reference spectra to address noise and other aspects found in spectral absorption data. Statistical techniques such as principal component analysis (PCA), partial least squares regression (PLSR), and the like may be utilized to extract relevant features and/or reduce the dimensionality of the spectra, enabling comparison of the sample spectra to the reference spectra. In effect, the method may compare sample spectra and reference spectra by identification of patterns between the sample spectra and reference spectra. For example, this may include review of the general shape of the spectra, amplitudes of peaks, and other characteristics of the spectra. For the purposes of this disclosure, the reference spectra may be those of known pathogens.

In a further embodiment, the comparison of sample spectra and reference spectra may be automated through the use of machine learning algorithms or other related means. In such an embodiment, models may be trained on a dataset of known spectra (e.g., derived from a pathogen encyclopedia or other database), wherein the system may classify or quantify the degree of similarity between sample spectra and reference spectra. Various machine learning methods may be implemented, including, but not limited to, deep learning architectures, such as convolutional neural networks (CNNs) or recurrent neural networks (RNNs).

The present disclosure comprises implicit steps of categorizing viruses into distinct types and subtypes, when such types or subtypes exist and/or are able to be derived from the available sample spectra and reference spectra. These types and subtypes may be evaluated based on their genetic characteristics, for example, as differentiated via MIR analysis. Viruses, as a class, may demonstrate diversity in their genome sequences, manifesting as distinct types and subtypes. Such types and subtypes may include different biological properties, which may induce varying degrees of symptoms or illness in a patient. Yet further, certain types or subtypes may be more or less resistant to certain drugs or treatments. By categorizing viruses into types and/or subtypes, the method described herein may enhance the system's ability to assess pathogenicity, thereby improving diagnosis and treatment selection.

### Example 1

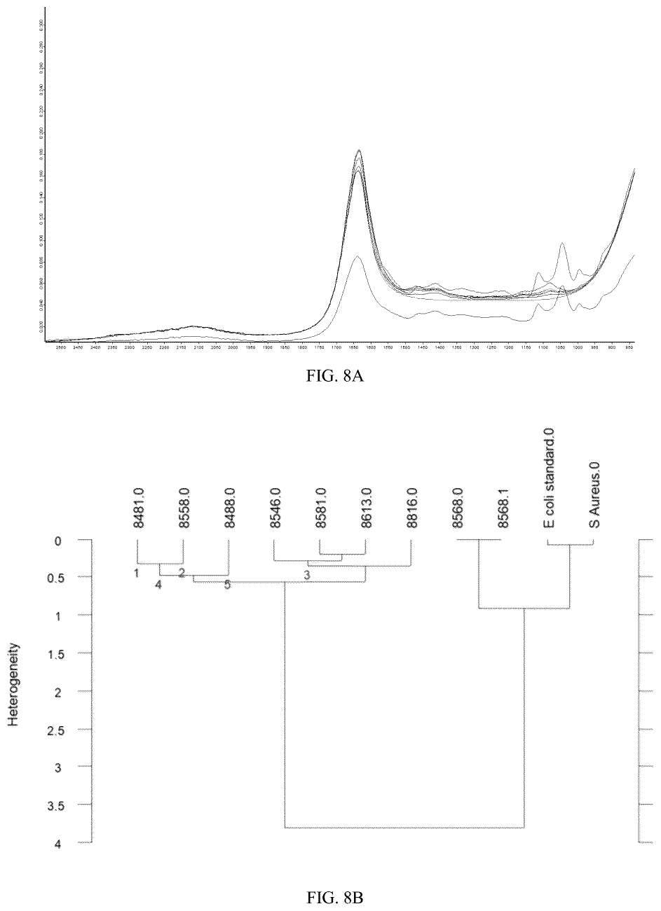

One example of the system and method is described herein. In this example, 12 samples, six comprising unknown pathogens (8558, 8481, 8488, 8551, 8613, 8568, 8465, 8546, 8819) information and three control samples (E-coli, S. Aureus and water) having known pathogens or water, were tested to determine if the system and method could differentiate between pathogens in the samples. Each of the samples were processed using FT-IR spectrometry and the spectra for all of the samples is illustrated in FIG. 8A.

A heterogeneity analysis of the spectra for all of the samples was conducted and is illustrated in the dendrogram shown in FIG. 8B. The sample 8568 was analyzed twice and was determined to have a heterogeneity of 0, which indicates they are identical. This determination is in accordance with the expected results and demonstrates an ability of the system to receive a consistent result.

The next closest groupings are the two bacterial controls (E-coli, S. Aureus), which have heterogencity of approximately 0.1. This indicates how close the spectra of those two bacteria are to one another. Although this is a small number, it demonstrates that they do differ from each other and this difference can be detected, despite the significant similarities between the samples. The other samples (8481, 8558, 8488,8546,8581,8613 and 8818) all have heterogencities of 0.2-0.6, from each other. This indicates that despite some similarities between the samples, the system is capable of detecting the various differences.

Thus, the system and method are suitable for determining differences, even minor differences, between samples. Further, the system and method are capable of identifying identical samples.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method for detecting pathogens in a sample comprising the steps of:
processing a sample using MIR spectrometry to identify sample spectra data;
detecting a presence of a pathogen in the sample spectra data, wherein the presence of the pathogen is detected according to a method comprising the steps of:
identifying the pathogen in the sample, wherein the pathogen is identified when a concentration of the pathogen in the sample is over a level of detection (LOD);
identifying a volume of the pathogen in the sample, wherein the volume is a level of quantification (LOQ) of the pathogen; and
determining the presence of a resistant gene in the pathogen; and
generating a report comprising the pathogen detected and the presence of resistant genes in the sample.

2. The method of claim 1, wherein the MIR spectrometry is FT-IR spectrometry.

3. The method of claim 1, wherein identifying the pathogen in the sample comprises comparing the sample spectra data with a plurality of pathogen profiles, each of the plurality of pathogen profiles corresponding to a known pathogen and comprising spectra data corresponding to the known pathogen.

4. The method of claim 3, wherein comparing the sample spectra data with the plurality of pathogen profiles comprises determining a best fit pathogen profile to the sample spectra data.

5. The method of claim 1, wherein identifying the volume of the pathogen in the sample comprises determining a concentration of the pathogen in sample.

6. The method of claim 1, wherein when the presence of the resistant gene is determined, the method further comprises the step of correlating a protein structure of the resistant gene with a database to determine an antibiotic resistance of the pathogen.

7. A method for detecting pathogens in a sample comprising the steps of:
processing a sample using an FT-IR spectrometer to identify sample spectra data, wherein the sample spectra data comprises an absorption spectrum of the sample;
detecting a presence of a pathogen in the sample spectra data, wherein the presence of the pathogen is detected according to a method comprising the steps of:
identifying the pathogen in the sample, wherein identifying the pathogen in the sample comprises comparing the sample spectra data with a plurality of pathogen profiles, stored in a database, each of the plurality of pathogen profiles corresponding to a known pathogen and comprising spectra data corresponding to the known pathogen;
identifying a concentration of the pathogen in the sample; and
determining the presence of a resistant gene in the pathogen from the absorption spectrum of the sample and the database to determine an antibiotic resistance of the pathogen; and
generating a report comprising the pathogen detected and the presence of resistant genes in the sample.

8. The method of claim 7, wherein the absorption spectrum of the sample is a compared to an absorption spectrum of the known pathogen in the plurality of pathogen profiles to determine a best fit, by comparing any of a frequency, amplitude, or spread of the absorption spectrums.

9. The method of claim 7, wherein the concentration of the pathogen in the sample corresponds to any of a high colonization, a medium colonization, or a low colonization of the pathogen.

10. The method of claim 9, wherein when the concentration of the pathogen is the high colonization, the report comprises to a recommendation of treatment.

11. The method of claim 9, wherein the high colonization corresponds to at least 100,000 CFU per ml, the medium colonization corresponds to 50,000-100,000 CFU per ml, and the low colonization corresponds to 10,000-50,000 CFU per ml.

12. The method of claim 7, wherein when the presence of the resistant gene is determined, the method further comprises correlating a protein structure of the resistant gene with a database to determine an antibiotic resistance of the pathogen.

13. The method of claim 7, wherein identifying the pathogen comprises identifying the presence of the pathogen greater than a level of detection (LOD).

14. The method of claim 7, wherein the step of identifying the concentration of the pathogen in the sample further comprises comparing the sample spectra data with the plurality of pathogen profiles, stored in the database, wherein each of the plurality of pathogen profiles comprise concentration-specific spectra, wherein each of the concentration-specific spectra are determined by using the FT-IR spectrometer with reference samples of varying concentrations.

15. The method of claim 7, wherein the plurality of pathogen profiles comprises pathogen profiles of subtypes.
